(19) United States
(12) Patent Application Publication
(10) Pub. No.: US 2006/0258568 A1
(43) Pub. Date: Nov. 16, 2006

(54) Use of human chorionic gonadotropin in the treatment in the treatment of symptoms endometriosis

(75) Inventor: Ambros Valentin Huber, Vienna (AT)

Correspondence Address: FULBRIGHT & JAWORSKI L.L.P., 600 CONGRESS AVE., SUITE 2400, AUSTIN, TX, 78701, US

(73) Assignee: Austia Wirtschaftsservice Gesellschaft mit Beschrankter, Vienna (AT)

(21) Appl. No.: 10/544,856

(22) PCT Filed: Feb. 7, 2003
(86) PCT No.: PCT/IB03/01066

§ 371 Date: Jun. 5, 2006

(51) Int. Cl. A61K 38/24

(52) U.S. Cl. 514/8

(57) ABSTRACT

The human chorionic gonadotropin is found to be a new, efficient agent for the treatment of symptoms like chronic pelvic pain, pre-menstrual syndrom, dysmenorrhea, dyspareunia and infertility, caused by endometriosis.

### FIELD OF THE INVENTION

This invention relates to a second medical use of human chorionic gonadotropin in a new method for the therapeutic management of chronic and acute pelvic pain, pre-menstrual syndrom (PMS), dysmenorrhea, dyspareunia and infertility caused by endometriosis.

It was found by us that hCG, commonly used for the induction of ovulations and for the treatment of luteal insufficiency, also reduces the symptoms caused by endometriosis.

### BACKGROUND OF THE INVENTION AND PRIOR ART

Human chorionic gonadotropin (hCG) is a glycoprotein (molecular weight of about 36.700), that is composed of an α- and a β-subunit. It is secreted by the syncytiophoblast. The rate of secretion of hCG increases rapidly in the first few weeks of pregnancy, and maximal levels are attained in maternal blood and urine at approximately 10 weeks ad gestation.

The physiological role of hCG in human pregnancy is not fully defined. hCG acts as a luteotropin to maintain the corpus luteum and serves to convert the corpus luteum of menstruation to the corpus luteum of pregnancy through its capacity to stimulate the secretion of progesterone and relaxin.

It was found that hCG inhibits the growth of benign, borderline and malignant ovarian epithelial cell lines (Tourgeman et. al., 2002)

hCG and the hCG—subunit also inhibit the proliferation of cell lines derived from Kaposi's sarcoma (KS). Regression of Kaposi's sarcoma has been shown in two woman during pregnancy, where the level of this hormone is high (Lunardi-Iskandar et al., 1995).

Albini and Orengo (1996) found that KS cells membranes above defined contain binding sites of β-core and deglycosylated hCG and it was concluded that β-core itself and/or deglycosylated hCG is the agent responsible for the in vivo and in vitro biological activities found by Lunardi-Iskandar (1995).

Endometriosis is defined by the presence and proliferation of endometric tissue (glands and stromal) outside the endometrial cavity as well as in the myometrium. Acute or chronic pelvic pain, dysmenorrhea, dyspareunia, pre-menstrual syndrom and infertility perform the most frequent clinical symptoms. The ectopic endometric tissue responds to ovarian hormones undergoing cyclic changes. The cyclical bleeding from the endometric deposit contributes to a local inflammatory reaction. Endometriosis commonly affects women during their childbearing years.

Treatments of endometriosis include operative laparoscopy with resection or ablation of endometric implant, and temporary gonadal suppression by danazol, progestagens, continuous oral contraceptive tablets, or luteinizing hormone-releasing hormone (LHRH ) agonists.

### SUMMARY OF THE INVENTION

It was observed that in patients with endometriosis, by whom the endometric implants were removed by operative laparoscopy and who, due to persistent pain, consecutively were “down-regulated” with LHRH agonists and submitted to a temporary gonadal suppression by danazol and progestagens, the pain remained. I found that a treatment with hCG (Pregnyl®) injections resulted in an immediate and remaining improvement of the situation and in the following in a full disappearance of the pain.

### DETAILED DESCRIPTION OF THE INVENTION

Up until now, all allowed and commonly applied treatments of symptoms caused by endometriosis have no satisfying result.

Clinical experience showed that only a pregnancy can result in a permanent healing of these symptoms.

Based on these clinical experience, patients which have been submitted to all possible medical treatments of the symptoms caused by endometriosis and still were in pain, have been treated with hCG.

The hCG is administrated over a period of 3 days to 60 months, with a concentration of 1 I.E. to 500.000 I.E., every day, every 2^{nd}, 3^{rd}, 4^{th}, 5^{th}, 6^{th}, or 7^{th} day.

The hCG can be injected intramuscular or can be administrated orally.

According to the invention a hCG is administrated for 2 weeks every 3^{rd} day with a concentration of 750 I.E., consequetively for 2 weeks every 3^{rd} day with a concentration of 1.500 I.E., followed by 2 weeks every 4^{th} day with a concentration of 3.000 I.E., the following 2 weeks every 5^{th} day with a concentration of 5.000 I.E., the following 2 weeks every 4^{th} day with a concentration of 3.000 I.E., followed by 2 weeks every 3^{rd} day with a concentration of 1.500 I.E., and the last 2 weeks every 3^{rd} day with a concentration of 750 I.E. (Diagram1).

According to another aspect of the invention, the hCG is administrated at day 1 with a concentration of 750 I.E., at day 3 with a concentration of 1.000 I.E., at day 5 with a concentration of 1.500 I.E., at day 8 with a concentration of 1.750 I.E., at day 12 with a concentration of 2.000 I.E., at day 17 with a concentration of 5.000 I.E., at day 24 with a concentration of 7.500 I.E., at day 31 with a concentration of 10.000 I.E., at day 38 with a concentration of 5.000 I.E., at day 48 with a concentration of 2.500 I.E., at day 55 with a concentration of 2.000 I.E., the following 4 weeks with a concentration of 1.500 I.E. once a week, followed by 4 weeks with a concentration of 1.000 I.E. once a week and finally followed by 4 weeks with a concentration of 750 I.E. once a week (Diagram 2).

According to another aspect of the invention, the hCG is administrated for 2 weeks every 3^{rd} day with a concentration of 750 I.E.. with a concentration of 1.500 I.E. every 3^{rd} day in the following 2 weeks followed by an administration with a concentration of 3.000 I.E. every 3^{rd} day for 2 weeks, followed by an administration with a concentration of 5.000 I.E. every 3^{rd} day for the following 2 weeks, during the following 2 weeks with a concentration of 3.000 I.E. every 3^{rd} day, with a concentration of 1.500 I.E. every 3^{rd} day for the following 12 weeks, followed by administration with a concentration of 750 I.E. 2 times a week for a period of 30 weeks (Diagram 3).

According to another aspect of the invention, the hCG is administrated for a period of 4 weeks with a concentration of 750 I.E.-5.000 I.E. every 2^{nd}, 3^{rd}, or 4^{th} day, followed by a period of 4 weeks with an administration every 2^{nd}, 3^{rd} or 4 day of a concentration of 1.500 I.E.-10.000 I.E., followed by an administration of a concentration of 1.000 I.E.-5.000 I.E. every 3^{rd} of 4^{th} day over a period of 10 months. Preferably, the hCG is administrated for a period of 4 weeks with a concentration of 750 I.E.-2.500 I.E. every 3^{rd} or 4^{th} day, followed by a period of 4 weeks with an administration of 1.500 I.E.-5.000 I.E. every 3^{rd} or 4^{th} day, followed by an administration of 1.000 I.E.-2.500 I.E. every 3^{rd} or 4^{th} day over a period of 10 months. More preferably, the hCG is administrated for a period of 4 weeks with a concentration of 750 I.E. every 4^{th} day, followed by a period of 4 weeks with an administration of 1.500 I.E. every 4^{th} day, followed by an administration of 1.000 I.E. every 4^{th} day over a period of 10 months.

According to another aspect of the invention, in analogy to the situation during pregnancy the hCG is administrated over a period of 9 months whereby the hCG is administrated once or twice a week in week 1 to 4 week with a concentration of 100 I.E.-2.000 I.E., in week 5-6 with a concentration of 2.500 I.E.-3.500 I.E., in week 7-8 with a concentration of 4.000 I.E.-20.000 I.E., in week 9-10 with a concentration of 30.000 I.E.-100.000 I.E., in week 11-14 with a concentration of 5.000 I.E-30.000 I.E., in week 15-20 with a concentration of 1.000 I.E.-3.000 I.E., in week 21-40 with a concentration of 100 I.E.-500 I.E. (Diagram 4).

According to another aspect of the invention, the hCG is administrated continuously as long as the patient suffers from pain, whereby the therapy is restricted to a period of 4 weeks to 5 years, every day with a concentration of 1 I.E.-500 I.E., whereby in case of acute pain the concentration of the hCG administrated is augmented to 5,000-500.000 I.E. for a period of 1, 2, 3, 4, or 5 days, period in which the hCG is administrated every day or every 2^{nd} day.

According to another aspect of the invention, for the treatment of PMS and dysmenorrhea the hCG is administrated 3 days before menstruation and/or during menstruation with a concentration of 750 I.E.-5.000 I.E. every day. Preferably a concentration of 1.500 I.E. is administrated every day.

### LITERATURE

Albini A, Paglieri I, Orengo G, Carlone S, Aluigi M G, DeMarchi R, Matteucci C, Mantovani A, Carozzi F, Donini S, Benelli R, 1997, “The beta-core fragment of human chorionic gonadotrophin inhibits growth of Kaposi's sarcoma-derived cells and a new immortalized Kaposi's sarcoma cell line.”, AIDS May 1997;11(6):713-21.
Tourgeman D E, Lu J J, Boostanfar R, Amezcua C, Felix J C, Paulson R J, “Human chorionic gonadotropin suppresses ovarian epithelial neoplastic cell proliferation in vitro.”, Fertil Steril November 2002;78(5):1096-9.
Lunardi-Iskandar Y, Bryant J L, Zeman R A, Lam V H, Samaniego F, Besnier J M, Hermans P, Thierry A R, Gill P, Gallo R C, “Tumorigenesis and metastasis of neoplastic Kaposi's sarcoma cell line in immunodeficient mice blocked by a human pregnancy hormone.” Nature May 4, 1995;375(6526):64-8.

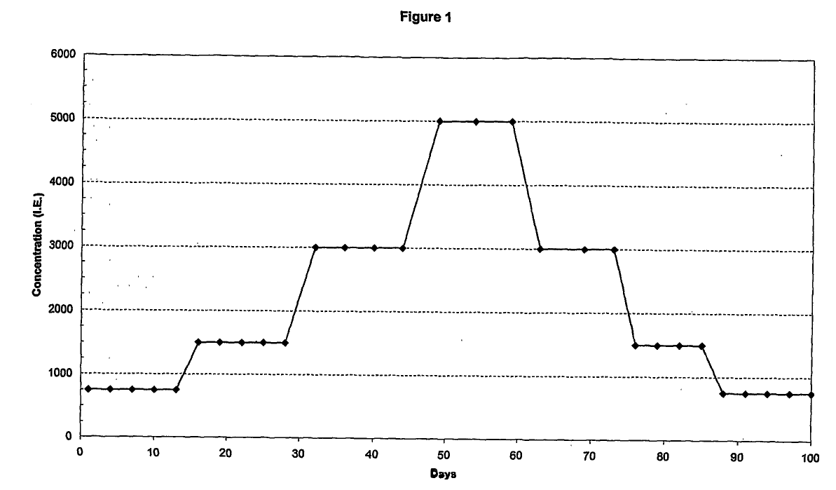

FIG. 1.: Change of the concentration of the medicament administrated with time.

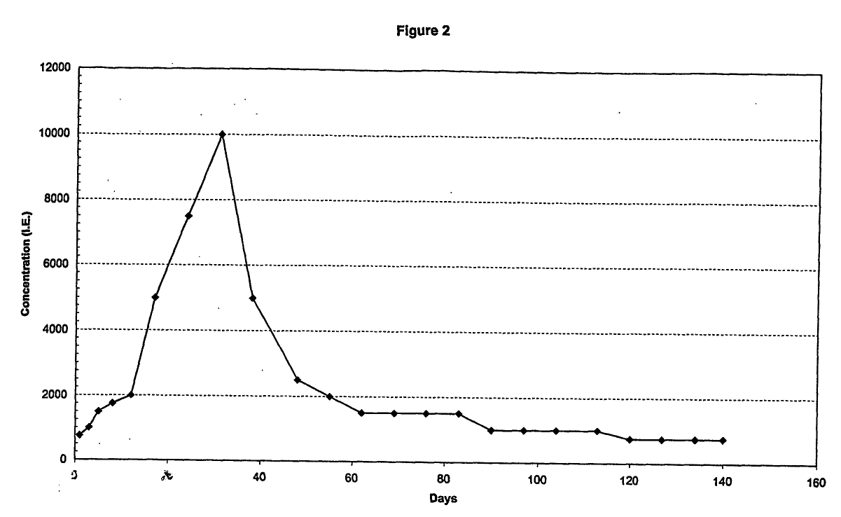

FIG. 2.: Change of the concentration of the medicament administrated with time.

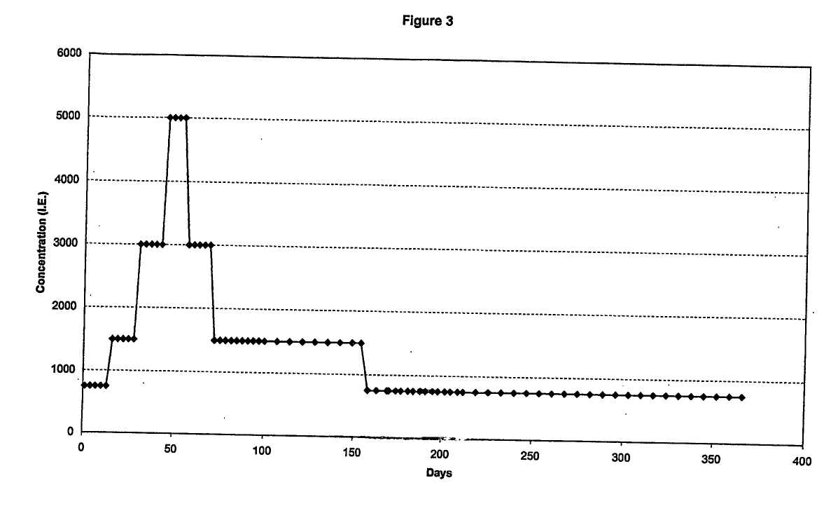

FIG. 3.: Change of the concentration of the medicament administrated with time.

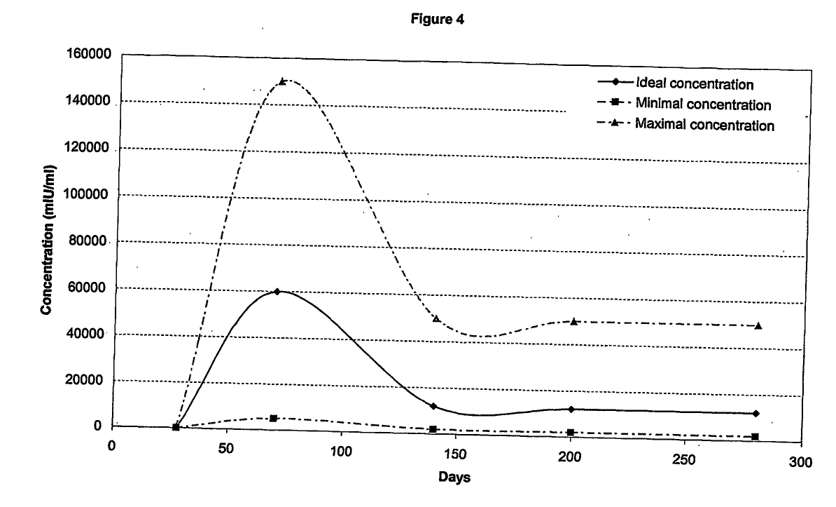

FIG. 4.: Change of the ideal, maximal and minimal concentration of the medicament administrated with time

1.-14. (canceled)

15. A method comprising:
obtaining human chorionic gonadotropin (hCG); and
administering the hCG to a patient having endometriosis.

16. The method of claim 15, wherein the patient exhibits at least one of the following symptoms of endometriosis: chronic pelvic pain, pre-menstrual syndrome (PMS), dysmenorrhea, or dyspareunia.

17. The method of claim 15, wherein the hCG is administered by intramuscular injection.

18. The method of claim 15, wherein the hCG is administered orally.

19. The method of claim 15, wherein the hCG is administered over a period of 3 days to 60 months.

20. The method of claim 15, wherein the hCG is administered at a concentration of 1 I.E. to 500,000 I.E.

21. The method of claim 15, wherein the hCG is administered every day, every 2^{nd}, 3^{rd}, 4^{th}, 5^{th}, 6^{th}, or 7^{th} day.

22. The method of claim 15, wherein the hCG is administered by intramuscular injection or by oral administration, over a period of 3 days to 60 months, at a concentration of 1 I.E. to 500,000 I.E., and every day, every 2^{nd}, 3^{rd}, 4^{th}, 5^{th}, 6^{th}, or 7^{th} day.

23. The method of claim 15, wherein the hCG is administered for 2 weeks every 3^{rd} day at a concentration of 750 I.E., consecutively for 2 weeks every 3^{rd} day at a concentration of 1,500 I.E., followed by 2 weeks every 4^{th} day at a concentration of 3,000 I.E., the following 2 weeks every 5^{th} day at a concentration of 5,000 I.E., the following 2 weeks every 4^{th} day at a concentration of 3,000 I.E., followed by 2 weeks every 3^{rd} day at a concentration of 1,500 I.E., and the last 2 weeks every 3^{rd} day at a concentration of 750 I.E.

24. The method of claim 15, wherein the hCG is administered at day 1 at a concentration of 750 I.E., at day 3 at a concentration of 1,000 I.E., at day 5 at a concentration of 1,500 I.E., at day 8 at a concentration of 1,750 I.E., at day 12 at a concentration of 2,000 I.E., at day 17 at a concentration of 5,000 I.E., at day 24 at a concentration of 7,500 I.E., at day 31 at a concentration of 10,000 I.E., at day 38 at a concentration of 5,000 I.E., at day 48 at a concentration of 2,500 I.E., at day 55 at a concentration of 2,000 I.E., the following 4 weeks at a concentration of 1,500 I.E. once a week, followed by 4 weeks at a concentration of 1,000 I.E. once a week, and finally followed by 4 weeks at a concentration of 750 I.E. once a week.

25. The method of claim 15, wherein the hCG is administered for 2 weeks every 3^{rd} day at a concentration of 750 I.E., at a concentration of 1,500 I.E. every 3^{rd} day in the following 2 weeks, followed by an administration at a concentration of 3,000 I.E. every 3^{rd} day for 2 weeks, followed by an administration at a concentration of 5,000 I.E. every 3 day for the following 2 weeks, during the following 2 weeks at a concentration of 3,000 I.E. every 3^{rd} day, at a concentration of 1,500 I.E. every 3^{rd} day for the following 12 weeks, followed by administration at a concentration of 750 I.E. 2 times a week for a period of 30 weeks.

26. The method of claim 15, wherein the hCG is administered for a period of 4 weeks at a concentration of 750 I.E.-5,000 I.E. every 2^{nd}, 3^{rd}, or 4^{th} day, followed by a period of 4 weeks with an administration every 2^{nd}, 3^{rd} or 4^{th} day of 1.500 I.E.-10,000 I.E., followed by an administration of 1.000 I.E.-5,000 I.E. every 3^{rd} of 4^{th} day over a period of 10 months.

27. The method of claim 26, wherein the hCG is administered for a period of 4 weeks at a concentration of 750 I.E.-2,500 I.E. every 3^{rd} or 4^{th} day, followed by a period of 4 weeks with an administration of 1,500 I.E.-5,000 I.E. every 3 or 4^{th} day, followed by an administration of 1,000 I.E.-2,500 I.E. every 3^{rd} or 4^{th} day over a period of 10 months.

28. The method of claim 27, wherein the hCG is administered for a period of 4 weeks at a concentration of 750 I.E. every 4^{th} day, followed by a period of 4 weeks with an administration of 1,500 I.E. every 4^{th} day, followed by an administration of 1,000 I.E. every 4^{th} day over a period of 10 months.

29. The method of claim 15, wherein the hCG is administered over a period of 9 months, and further wherein it is administered once or twice a week in weeks 1-4 at a concentration of 100 I.E.-2,000 I.E., in weeks 5-6 at a concentration of 2,500 I.E.-3,500 I.E., in weeks 7-8 at a concentration of 4,000 I.E.-20,000 I.E., in weeks 9-10 at a concentration of 30,000 I.E.-100,000 I.E., in weeks 11-14 at a concentration of 3,000 I.E.-30,000 I.E., in weeks 15-20 at a concentration of 1,000 I.E.-3,000 I.E., in weeks 21-40 at a concentration of 100 I.E.-500 I.E.

30. The method of claim 15, wherein the hCG is administered continuously as long as the patient suffers from pain, and further wherein the therapy is restricted to a period of 4 weeks to 5 years, every day at a concentration of 1 I.E.-500 I.E., and wherein in case of acute pain the concentration of the hCG administered is augmented to 5,000-500,000 I.E. for a period of 1, 2, 3, 4, or 5 days, during which the hCG is administered every day or every 2^{nd} day.

31. The method of claim 15, wherein the patient exhibits at least one of PMS or dysmenorrhea, and the hCG is administered approximately 3 days before menstruation at a concentration of 750 I.E.-5,000 I.E. every day.

32. The method of claim 31, wherein the hCG is administered every day at a concentration of 1,500 I.E.

33. The method of claim 31, wherein the hCG is further administered during menstruation at a concentration of 750 I.E.-5,000 I.E. every day.

34. The method of claim 33, wherein the hCG is administered every day at a concentration of 1,500 I.E.
